SEQUENCE LISTING (23 sequences)

SEQ ID NO: 1 - DNA, 20 bp, Artificial sequence
  Primer
caggggaagcaaaggagaag

SEQ ID NO: 2 - DNA, 20 bp, Artificial sequence
  Primer
cgactttcatagccctggac

SEQ ID NO: 3 - DNA, 23 bp, Artificial sequence
  Primer
cctgcatcctgtctggaagttag

SEQ ID NO: 4 - DNA, 21 bp, Artificial sequence
  Primer
aaagaatcattcaaccagcgg

SEQ ID NO: 5 - DNA, 22 bp, Artificial sequence
  Primer
gactgggagatatggccacatg

SEQ ID NO: 6 - DNA, 23 bp, Artificial sequence
  Primer
gagactcataatgcttggttcag

SEQ ID NO: 7 - DNA, 35 bp, Artificial sequence
  EMSA probe corresponding to -879 to -845bp of the TNF promoter
gagtatggggacccccccttaacgaagacagggcc

SEQ ID NO: 8 - DNA, 1861 bp, Homo sapiens
ctgaaccatccctgatgtctgtctggctgaggatttcaagcctgcctaggaattcccagcccaaagctgttggtcttgtccaccagctaggtggggcctagatccacacacagaggaagagcaggcacatggaggagcttgggggatgactagaggcagggaggggactatttatgaaggcaaaaaaattaaattatttatttatggaggatggagagaggggaataatagaagaacatccaaggagaaacagagacaggcccaagagatgaagagtgagagggcatgcgcacaaggctgaccaagagagaaagaagtaggcatgagggatcacagggccccagaaggcagggaaaggctctgaaagccagctgccgaccagagccccacacggaggcatctgcaccctcgatgaagcccaataaacctcttttctctgaaatgctgtctgcttgtgtgtgtgtgtctgggagtgagaacttcccagtctatctaaggaatggagggagggacagagggctcaaagggagcaagagctgtggggagaacaaaaggataagggctcagagagcttcagggatatgtgatggactcaccaggtgaggccgccagactgctgcaggggaagcaaaggagaagctgagaagatgaaggaaaagtcagggtctggaggggcgggggtcagggagctcctgggagatatggccacatgtagcggctctgaggaatgggttacaggagacctctggggagatgtgaccacagcaatgggtaggagaatgtccagggctatgaaagtcgagtatggggacccccmcttaaygaagacagggccatgtagagggccccagggagtgaaagagcctccaggacctccaggtatggaatacaggggacgtttaagaagatatggccacacactggggccctgagaagtgagagcttcatgaaaaaaatcagggaccccagagttccttggaagccaagactgaaaccagcattatgagtctccgggtcagaatgaaagaagagggcctgccccagtggggtctgtgaattcccgggggtgatttcactccccggggctgtcccaggcttgtccctgctacccgcacccagcctttcctgaggcctcaagcctgccaccaagcccccagctccttctccccgcagggcccaaacacaggcctcaggactcaacacagcttttccctccaaccccgttttctctccctcaacggactcagctttctgaagcccctcccagttctagttctatctttttcctgcatcctgtctggaagttagaaggaaacagaccacagacctggtccccaaaagaaatggaggcaataggttttgaggggcatggggacggggttcagcctccagggtcctacacacaaatcagtcagtggcccagaagacccccctcggaatcggagcagggaggatggggagtgtgaggggtatccttgatgcttgtgtgtccccaatttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacacccagccagcagacgctccctcagcaaggacagcagaggaccagctaagagggagagaagcaactacagaccccccctgaaaacaaccctcagacgccacatcccctgacaagctgccaggcaggttctcttcctctcacatactgacccacggcttcaccctctctcccctggaaaggacaccatg

SEQ ID NO: 9 - DNA, 25 bp, Artificial sequence
  Primer
aatagatctggaagttttccgctgg

SEQ ID NO: 10 - DNA, 19 bp, Artificial sequence
  Primer
aatgccaagcttggaagag

SEQ ID NO: 11 - DNA, 27 bp, Artificial sequence
  Primer
aatggtaccccacagcaatgggtagga

SEQ ID NO: 12 - DNA, 27 bp, Artificial sequence
  Primer
aatagagctcggaggtcctggaggctc

SEQ ID NO: 13 - DNA, 28 bp, Artificial sequence
  Primer
aatggatccatgaacaatccgtcagaaa

SEQ ID NO: 14 - DNA, 25 bp, Artificial sequence
  Primer
aatctcgagctgtgccttggaggcg

SEQ ID NO: 15 - DNA, 24 bp, Artificial sequence
  Primer
aatctcgagtggtgggttgattct

SEQ ID NO: 16 - DNA, 24 bp, Artificial sequence
  Primer
aatctcgagtgagaggttctctgc

SEQ ID NO: 17 - DNA, 24 bp, Artificial sequence
  Primer
aatctcgaggggagtatcaattgg

SEQ ID NO: 18 - DNA, 39 bp, Artificial sequence
  Probe
agctgagtatggggacccccccttaaygaagacagggcc

SEQ ID NO: 19 - DNA, 38 bp, Artificial sequence
  Probe
gctggccctgtcttcrttaagggggggtccccatactc

SEQ ID NO: 20 - DNA, 24 bp, Artificial sequence
  Probe
agctcccttaaygaagacagggcc

SEQ ID NO: 21 - DNA, 24 bp, Artificial sequence
  Probe
agctggccctgtcttcrttaaggg

SEQ ID NO: 22 - DNA, 21 bp, Artificial sequence
  Probe
agctcccttaatgcaaatagg

SEQ ID NO: 23 - DNA, 21 bp, Artificial sequence
  Probe
agctcctatttgcattaaggg

(19) United States
(12) Patent Application Publication
(10) Pub. No.: US 2004/0219555 A1
(43) Pub. Date: Nov. 4, 2004

(54) Method of determining susceptibility to inflammatory bowel disease

(76) Inventor: David Van Heel, London (GB)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/492,113

(22) PCT Filed: Oct. 9, 2002
(86) PCT No.: PCT/GB02/04582

(30) Foreign Application Priority Data
Oct. 10, 2001 (GB) 0124315.3

(51) Int. Cl. C12Q 1/68

(52) U.S. Cl. 435/6

(57) ABSTRACT

The present invention relates to the identification of the TNF haplotype TNF-1031C/-857C/-863C/-308G as being associated with susceptibility to Crohn's Disease. The invention also relates to the identification of the -857C allele as associated with inflammatory Bowel Disease. Methods and means for determining susceptibility and preventing disease in a subject are also provided.

The present invention relates to methods for determining susceptibility to Inflammatory Bowel Disease, in particular Crohn's Disease, wherein the methods are based upon genotyping polymorphisms in the TNF gene. Also provided are means for carrying out the methods of the invention. In addition, the present invention provides methods and means for preventing or treating Inflammatory Bowel Disease, based upon modulating the activity of TNF transcription factors, OCT1 and NF-κB.

In recent years, it has been recognised that there is considerable genetic diversity in human populations, with common polymorphisms occurring on average at least every kilobase in the genome. Polymorphisms which affect gene expression or activity of the encoded gene product may account for susceptibility to, or expression of, disease conditions, either directly or through interaction with other genetic and environmental factors.

Understanding the molecular basis for disease, by sequencing the human genome and characterising polymorphisms, will enable the identification of those individuals at greatest risk of disease. This will allow the better matching of treatment and disease, and enable the production of new and improved targets for drugs. Screening and treatment of disease may also be better targeted to those in need, thus increasing the cost-effectiveness of health-care provision.

One area in need of such approaches is the diagnosis and treatment of inflammatory diseases. Inflammation, which can be broadly defined as the destructive sequelae to activation of elements of the body's immune system, is a feature of many diseases including infection, autoimmune disorders and benign and malignant hyperplasia The identification of genetic factors which influence susceptibility to such disorders will provide important new insights into inflammatory disease, and may yield important new diagnostic and/or prognostic tests and treatments.

Inflammatory Bowel Disease (IBD) is a chronic inflammatory disease of the bowel and gastrointestinal (GI) tract causing abdominal pain, diarrhoea and rectal bleeding. IBD can exist either as Ulcerative Colitis (UC), which affects the large bowel and rectum and is generally confined to the intestinal mucosa, or as Crohn's Disease (CD) which in contrast can affect any region of the GI tract, and may affect all layers of the gut (a transmural disease). Both UC and CD may affect other parts of the body, for example the skin and joints. UC and CD may also increase the risk of cancer. The symptoms of IBD are non-specific, and in around 10% of cases it is not possible to distinguish between UC and CD. These cases are classified as indeterminate.

Whilst incidence of the disease is about 0.1 to 0.2%, and higher in certain northern European populations and in Ashkenazi Jews, the precise mechanisms underlying susceptibility to the disease remain unclear. Preliminary research suggests that both genetic and environmental factors are involved, with chronic inflammation being the result of an autoimmune reaction. Research into the mechanism underlying IBD has focussed on the nature of the immune response in the intestinal mucosa. In normal subjects, the intestine is the site of tightly regulated inflammation, due to continued exposure to novel antigens and potentially pathogenic microorganisms. In IBD, this tightly regulated inflammation is thought to become de-regulated, with Th1-like T cells driving the resultant exaggerated inflammation. In support of this hypothesis, it has been shown that inhibition of Th-1 responses in acid mice reconstituted with CD45RBhi CD4⁺ T-cells inhibits IBD. Treatment of IBD at present includes administration of aminosalicylates (ASA) such as sulfasalzine and corticosteroids. The latter shows substantial long-term toxicity, and therefore immunosuppressive agents such as imuran and 6-mercaptopurine are useful in reducing the required dose of steroids. Other treatments include Metronidazole and nicotine in the treatment of CD and acute UC respectively, and the potent immunosuppressant cyclosporine and methotrexate. A more recent approach is the use of an anti-TNFα antibody,

Infliximab, Which is given as a single i.v. infusion of 5 mg/kg over 2 hours for moderate to severe CD subjects who are unresponsive to conventional treatment. However, the use of any immunosuppressive agent leads to side effects such as an increased risk of infection. Further, drug treatments are often only marginally effective and severe EBD usually leads to a need for surgery. Whilst this latter option can cure UC, it often leads to the need for an ileostomy, and cannot cure CD. EBD is therefore a significant medical problem, and though treatments are available, they show variable efficacy and often severe side effects.

An alternative approach to understanding IBD and finding new treatments has been to identify the genetic determinants underlying disease susceptibility. IBD is a highly familial disease, with a relative risk to siblings of affected individuals of between 7-10 for UC and 20-30 for CD, compared to the population risk. Genome wide scans have implicated regions on chromosomes 3, 6, 7 and 12 as being linked with IBD susceptibility.

TNF is a pro-inflammatory cytokine which plays an important role in the initiation and regulation of immune responses. TNF levels are elevated in the serum, mucosa and stool of IBD patients. Further evidence of its key role in intestinal inflammation is provided by animal models and by the therapeutic efficacy of anti-TNF monoclonal antibodies in human Crohn's Disease and Ulcerative Colitis. Increased TNF biosynthesis by deletion of the 3′ regulatory sequences of the TNF gene transcript in mice results in a Crohn's Disease like phenotype and mice made deficient in TNF show marked reduction in chemically induced intestinal inflammation.

In humans, transcription regulation of TNF is cell- and stimulus-specific and involves a variety of regulatory elements sited in the 5′ flanking region. A growing body of evidence indicates that NF-κB/Rel transcription factors are necessary for TNF gene activation in monocytes, and increased levels of TNF production and of NF-κB nuclear translocation have been shown in lamina propria monocytes derived from patients with IBD. TNF production is under strong genetic influence and polymorphic sites in the promoter region can affect transcription factor binding.

The present invention aims to improve the diagnosis and treatment of IBD patients by providing means and methods for the detection and treatment of individuals having, or being susceptible to, Inflammatory Bowel Disease, in particular Crohn's Disease and Ulcerative Colitis.

In a first aspect of the present invention, there is provided method of determining susceptibility of a Caucasian subject to Crohn's Disease, the method comprising screening the genetic material of the subject for the TNF -1031C/-863C/-857C/-308G haplotype.

The invention defined by the first aspect has been based upon the surprising discovery that this particular haplotype, or combination of alleles of the -1031T/C, -863C/A, -857C/T and -308G/A polymorphisms, is prevalent in Caucasian subjects having Crohn's Disease, and thus may be used as a tool for determining whether Caucasian subjects are susceptible to this particular disease by screening for this combination of alleles.

By determining susceptibility to disease is meant assessing whether a subject is likely to suffer from Inflammatory Bowel Disease in the future, and therefore susceptibility is preferably determined prior to the onset of symptoms. However, it is envisaged that the method of the present invention may be used to determine susceptibility to Inflammatory Bowel Disease, or to determine susceptibility to a particular form of Inflammatory Bowel Disease after the onset of symptoms but preferably before a clinical diagnosis can be made on the basis of such symptoms. Also envisaged is the use of the methods of the invention in confirming the diagnosis of a patient which has been made on the basis of clinical symptoms. This can provide greater accuracy of diagnosis, particularly for conditions where clinical diagnosis alone can be difficult, and therefore lead to faster and more accurate therapies.

In the context of the present invention, a Caucasian subject may be defined as a native of Europe, North Africa, Western and Central Asia, Australasia and America. Preferably, the term Caucasian excludes Japanese subjects.

The genetic material of the subject to be analysed may be DNA, or may be RNA or other options.

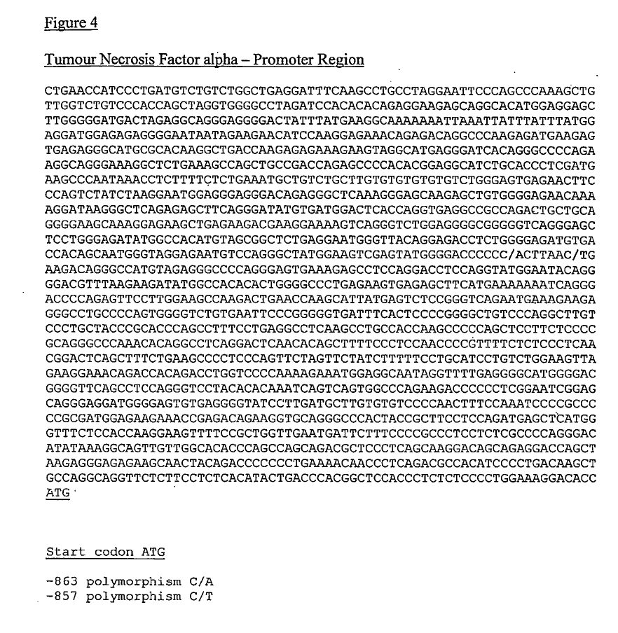

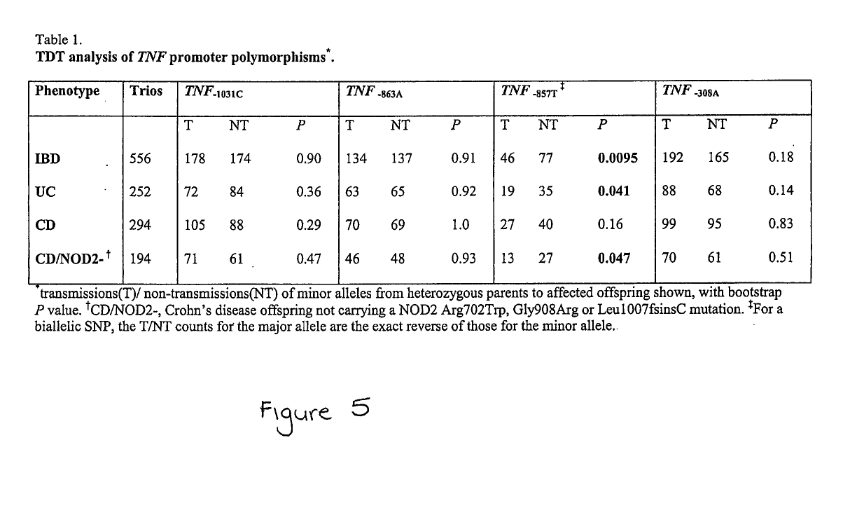

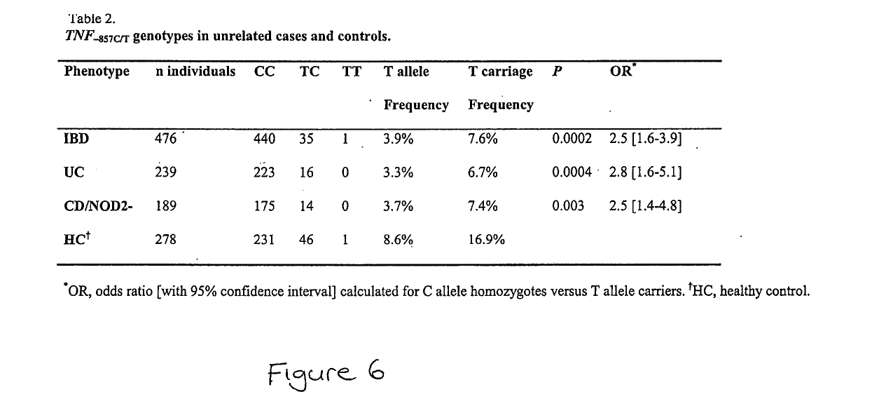

In the present invention, the TNF gene sequence is that detailed in Genbank Accession No Z15026 and part of the promoter region is shown in FIG. 4. The polymorphisms referred to in relation to the present invention have been given a positional reference with respect to this figure, wherein the nucleotide position 1 corresponds to the start codon ATG, indicated in FIG. 4. Nucleotides upstream of this are given a negative prefix. The sequence of FIG. 4, showing the −857 and −863 polymorphisms can be aligned with the sequence of Genbank or any other published sequence of TNF gene, to confirm the positions of the other polymorphisms. The polymorphisms which make up the haplotype of the invention are detailed in Wilson et al Hum Mol Genet 1992 August;1(5):353; Skoog et al Hum Mol Genet 1999 August;8(8):1443-9 and Higuchi et al Tissue Antigens 1998 June;51(6):605-12).

A polymorphism is typically defined as two or more alternative sequences, or alleles, of a gene in a population. A polymorphic site is the location in the gene at which divergence in sequence occurs. Examples of the ways in which polymorphisms are manifested include restriction fragment length polymorphisms, variable number of tandem repeats, hypervariable regions, minisatellites, di- or multi-nucleotide repeats, insertion elements and nucleotide deletions, additions or substitutions. The first identified allele is usually referred to as the reference allele, or the wild type.

Additional alleles are usually designated alternative or variant alleles. Haplotypes are the genotype of two or more polymorphisms combined.

A single nucleotide polymorphism, which in combination with others makes a haplotype, is a variation in sequence between alleles at a site occupied by a single nucleotide residue. Single nucleotide polymorphisms (SNP's) arise from the substitution, deletion or insertion of a nucleotide residue at a polymorphic site. Typically, this results in the site of the variant sequence being occupied by any base other than the reference base. Single nucleotide polymorphisms may result in corresponding changes to the amino acid sequence. For example, substitution of a nucleotide residue may change the codon, resulting in an amino acid change. Similarly, the deletion or insertion of three consecutive bases in the nucleic acid sequence may result in the insertion or deletion of an amino acid residue.

The method of the present invention is preferably carried out in vitro, on a sample removed from a subject. In such an embodiment, the invention does not define a method of medical treatment by diagnosis practiced on the human or animal body.

Any biological sample comprising cells containing nucleic acid or protein is suitable for this purpose. Examples of suitable samples include whole blood, semen, saliva, tears, buccal, skin or hair. For analysis of cDNA, mRNA or protein, the sample must come from a tissue in which the TNF gene is expressed, and thus it is preferable to use blood monocytes (preferably for RNA), blood serum (preferably for protein) and gut tissue biopsy samples.

Any method, including those known to persons skilled in the art, may be used to screen the genetic material of a subject according to the present invention. Examples of suitable methods include amplification, for example by PCR, followed by restriction enzyme digestion; southern blotting; allele specific amplification; RFLP analysis; direct probing; single base extension, minisequencing and MALDI-TOF based assay systems. In determining the haplotype of a subject, each polymorphism of the haplotype may be genotyped individually, and the results combined to determine the haplotype. Any of the afore-mentioned methods of genotyping may be appropriate to such an embodiment. Alternatively, the combination of polymorphisms may be genotyped simultaneously, using the above mentioned methods.

In determining the genotype of a subject according to the present invention, it may be desirable to use methods which enable simultaneous detection of multiple polymorphisms, for example, a diagnostic strip containing allele specific detection means such as probes or primers; or nucleic acid arrays, as described in WO95/11995. The array may contain a number of probes, each designed to identify one or more polymorphisms of the TNF gene, as described in WO95/11995.

The above described methods may require amplification of the genetic material from the subject, and this can be done by techniques known in the art, such as PCR (see PCR Technology: Principles and Applications for DNA Amplification (ed. H. A. Erlich, Freeman Press, NY 1992. Other suitable amplification methods include ligase chain reaction (LCR) (Wu et al., Genomics 4 560 (1989), transcription amplification (Kwoh et al., Proc Natl Acad Sci USA 86 1173 (1989)), self sustained sequence replication (Guatelli et al., Proc Natl Acad Sci USA 87 1874 (1990)) and nucleic acid based sequence amplification (NASBA). The latter two methods both involve isothermal reactions based on isothermal transcription which produce both single stranded RNA and double stranded DNA as the amplification products, in a ratio of 30 or 100 to 1, respectively.

In a second aspect of the invention, there is provided a method of confirming the diagnosis of a Caucasian subject as having Crohn's Disease, the method comprising screening genetic material of the subject for the presence of the TNF-1031 C/-863C/-857C/−308G haplotype. The method of this aspect may be useful where clinical symptoms of disease are present, but further information is required in order to confirm the diagnosis as being Crohn's Disease, or to distinguish from other diseases with similar symptoms.

In a third aspect of the invention, there is provided a kit for use in determining the susceptibility of a Caucasian subject to Crohn's Disease, the kit comprising means for screening for the TNF -1031C/-863C/-857C/-308G haplotype, and a key indicating the correlation between genotype and susceptibility to Crohn's Disease. Preferably, the key will be in the form of a chart or similar, indicating the correlation between each allele of each polymorphism, and each possible haplotype, with the degree of susceptibility to disease.

Preferably, the kit comprises any means suitable for screening genetic material of a subject for the above mentioned haplotype, which may be means suitable for carrying out any of the methods exemplified above. In a preferred embodiment, the kit may comprise primers for amplification of a portion of the TNF gene. Suitable primers for each polymorphisms of the above haplotype are:

| | | |
|---|---|---|
| TNF-1031T/C: | Forward | 5′-CAGGGGAAGCAAAGGAGAAG-3′ |
| | Reverse | 5′-CGACTTTCATAGCCCTGGAC-3′ |
| TNF-308G/A: | Forward | 5′-CCTGCATCCTGTCTGGAAGTTAG-3′ |
| | Reverse | 5′-AAAGAATCATTCAACCAGCGG-3′ |

The following primers can be used for both TNF -857C/T and TNF -863C/A:

| | |
|---|---|
| Forward | 5′-GACTGGGAGATATGGCCACATG-3′ |
| Reverse | 5′-GAGACTCATAATGCTTGGTTCAG-3′ |

The kit may additionally comprise means for performing the screening procedure, such as Taq polymerase, restriction enzymes, labels and buffers.

In a fourth aspect of the invention, there is provided a method of preventing and/or treating Crohn's disease in a Caucasian subject, the method comprising introducing into the subject genetic material comprising the TNF -1031T allele and/or the TNF 863T allele and/or the TNF -857T allele, and/or the TNF -308A allele. Preferably, genetic material comprising the TNF -1031C/-863T/-857T/-308A haplotype is introduced. Preferably, the genetic material introduced into the subject comprises a portion of the TNF gene including the relevant allele. The genetic material is therefore at least 30 nucleotides in length, more preferably at least 50, 70, 80, 100, 150, or 200 nucleotides in length. In a most preferred embodiment, the genetic material introduced will comprise the TNF gene, such that TNF may be produced from the foreign genetic material, or the genetic material introduced will be capable of recombining with the naive TNF gene, thus having the effect of replacing part of the gene responsible for abnormal regulation or processing of the gene. Methods and means to achieve homologous recombination in vivo will be known to persons skilled in the art.

Preferably, the subject has first been determined as being susceptible to Crohn's Disease, preferably using the method of the first aspect of the invention.

Any suitable means for introduction of genetic material may be used, including suitable gene therapy methods known in the art. In general, genetic material may be introduced into the target cells of a subject, usually in the form of a vector and preferably in the form of a pharmaceutically acceptable carrier. Any suitable delivery vehicle may be used, including viral vectors, such as retroviral vector systems which can package a recombinant genome. The retrovirus could then be used to infect and deliver the polynucleotide to the target cells. Other delivery techniques are also widely available, including the use of adenoviral vectors, adeno-associated vectors, lentiviral vectors, pseudotyped retroviral vectors and pox or vaccinia virus vectors. Liposomes may also be used, including commercially available liposome preparations such as Lipofectin®, Lipofectamine®, (GIBCO-BRL, Inc. Gaitherburg, Md.), Superfect® (Qiagen Inc, Hilden, Germany) and Transfectam® (Promega Biotec Inc, Madison Wis.).

The genetic material may be operably linked to one or more regulatory elements including a promoter; regions upstream or downstream of a promoter such as enhancers which regulate the activity of the promoter; an origin of replication; appropriate restriction sites to enable cloning of inserts adjacent to the genetic material; markers, for example antibiotic resistance genes; ribosome binding sites: RNA splice sites and transcription termination regions; polymerisation sites; or any other element which may facilitate the cloning and/or expression of the genetic material. A preferred marker for use in the present invention is the fatty acid binding protein gene (Fabp), as detailed in Saam et al J Biol Chem 274(53): 38071-82 (1999). The sequence may comprise a 3′ polyadenylation site.

Appropriate regulatory elements, in particular, promoters will usually depend upon the host cell into which the expression vector is to be inserted. Where microbial host cells are used, promoters such as the lactose promoter system, tryptophan (Trp) promoter system, β-lactamase promoter system or phage lambda promoter systems are suitable. Where yeast cells are used, preferred promoters include alcohol dehydrogenase I or glycolytic promoters. In mammalian host cells, preferred promoters are those derived from immunoglobulin genes, SV40, Adenovirus, Bovine Papilloma virus etc. Suitable promoters for use in various host cells would be readily apparent to a person skilled in the art (See, for example, Current Protocols in Molecular Biology Edited by Ausubel et al, published by Wiley).

The genetic material may be administered parenterally (eg, intravenously), transdermally, by intramuscular injection, topically or the like. Local administration of viral and/or liposome mediated delivery systems are preferred for use in the present invention. The exact amount of genetic material to be administered will vary from subject to subject and will depend upon age, weight, general condition, and severity or mechanism of the disorder.

In an alternative embodiment of the fourth aspect, there is provided the use of genetic material comprising the TNF -1031T allele and/or the TNF -863T allele and/or the TNF -857T allele, and/or the TNF -308A allele, in the manufacture of a medicament for the prevention or treatment of Crohn's disease in a Caucasian subject. Alternatively, the genetic material for use in manufacture of a medicament may comprise the TNF -1031T/-863T/-857T/-308A haplotype.

In a fifth aspect of the invention, there is provided a method of determining the susceptibility of a subject to Inflammatory Bowel Disease, the method comprising screening the genetic material of the subject to determine which allele of the TNF -857C/T polymorphism is present. This aspect of the invention is based upon the discovery that susceptibility to Inflammatory Bowel Disease is increased in those subjects having the C allele of this polymorphism.

In a preferred embodiment, the method of the fifth aspect is useful in determining susceptibility to Ulcerative Colitis or Crohn's Disease. Preferably, the subject is Caucasian.

In a further preferred embodiment of this aspect, the method may further comprising screening the genetic material of the subject to determine which allele of one or more of the NOD2 polymorphisms is present. It has been observed that the NOD2 and TNF -857 alleles act independently in conferring susceptibility to Inflammatory Bowel Disease, and for this reason it may be preferable to first determine whether the subject in question has one or more of the NOD2 variants which are associated with susceptibility to Inflammatory Bowel Disease. The NOD2 variants in question are described in Hugot et al (Nature 411 (6837): 599-603 (2001). The method of the fifth aspect is therefore preferably performed on subjects identified as lacking the NOD2 variants associated with susceptibility to Inflammatory Bowel Disease.

The preferred embodiments and methodology described in relation to the first aspect apply to this aspect mutatis mutafldis.

There is also provided a method for determining the response of a patient to treatment, the method comprising screening the genetic material of a subject to determine which allele of the TNF-857C/T and/or NOD2 polymorphisms are present. Thus, the underlying cause of the disease may be established by determining which polymorphism is present, and the appropriate treatment or preventative measure may be taken. For example, it may not be appropriate to administer to a subject having one or more of the NOD2 susceptibility allele(s) therapy based upon altering regulation or expression of the TNF gene, for example as described herein. The opposite may also be true.

In a sixth aspect of the invention, there is provided a kit for use in a method according to the fifth aspect, the kit comprising means for determining which allele of the TNF -857C/T and/or NOD2 polymorphisms are present, and a key correlating the presence of an allele with the susceptibility to disease. The preferred embodiments of the third aspect apply to this aspect mutatis mutandis.

In a seventh aspect of the invention, there is provided a method of preventing and/or treating Inflammatory Bowel Disease in a subject, the method comprising introducing into the subject genetic material comprising the TNF -857T allele.

In an alternative embodiment of the seventh aspect, there is provided the use of genetic material comprising the TNF -857T allele in the manufacture of a medicament for the prevention or treatment of Inflammatory Bowel Disease in a subject.

Preferably, the disease in Ulcerative Colitis or Crohn's Disease, and more preferably the subject is Caucasian. The other preferred embodiments and methodology of the fourth aspect apply here mutatis mutandis.

In a further aspect of the present invention, there is provided a method of treating a subject determined as being susceptible to disease, comprising administering an agent capable of preventing TNF production.

This aspect of the invention is based upon the inventors' discovery that presence of the -857C allele hinders binding of the TNF transcription factor, OCT1, to the TNF regulatory sequence. OCT1 has been shown to interact with the TNF transcription factor NF-κB.

The agent of this aspect is preferably one which is capable of modulating the activity of OCT1 and/or NF-κB. For example, the agent may modulate OCT1 to enable it to bind to the TNF−857C allele, thus enabling normal interaction with NF-κB and thus normal TNF production. Alternatively, the agent may modulate the interaction between OCT1 and NF-κB, for example by enabling OCT1 to interact with NF-κB without having to bind DNA at the TNF−857C allele.

A preferred agent for use in this aspect is one which is able to bind to the TNF -857C allele and also mediate interaction with NF-κB in order to regulate TNF production in a normal manner. Such an agent is preferably capable of interacting with the Rel homology domain of NF-κB, as detailed in Kieren et al (Cell 62(5) 1007-1018 (1990). such an agent is likely therefore to have a POU domain, as detailed in Herr et al Genes Dev 9(14):1679-1693 (1995). More preferably, the agent of this aspect by interacting with NF-κB is able to inhibit the activity of this latter transcription factor. The agent may be a agent is a variant of OCT1.

Preferably, the subject has been identified as being susceptible to Inflammatory Bowel Disease according to one or more of the previous aspects of the invention. More preferably, the subject suffers from, or is susceptible to, Ulcerative Colitis or Crohn's Disease.

In an alternative embodiment, there is provided the use of an agent capable of preventing TNF production in the manufacture of a medicament for use in treating a subject for Inflammatory Bowel Disease, in particular Ulcerative Colitis or Crohn's Disease.

The preferred embodiments of each aspect apply to the other aspects of the invention, mutatis mutandis.

The present invention will now be described by way of a non-limiting example, with reference to the following figures in which:

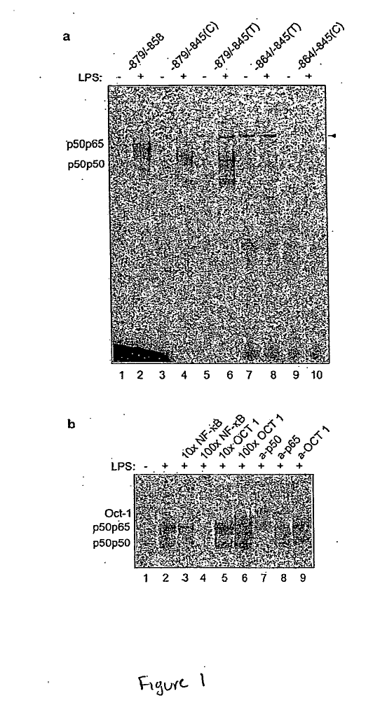

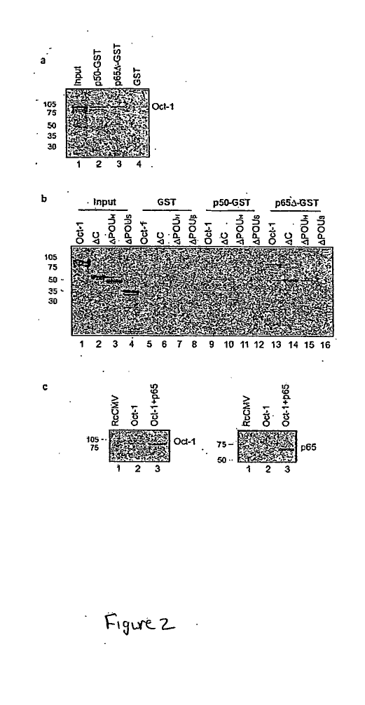

FIG. 1

Nuclear factors binding to TNF -857C and TNF -857T variants. (a) Nuclear extracts from MonoMac6 cells prior to LPS stimulation or after they have been stimulated for 1 h, were used in EMSA with radiolabelled probes corresponding to -879/858 nt of the TNF promoter (lanes 1, 2); -879/-845 nt (lanes 3, 4 for TNF-857C allele and lanes 5, 6 for TNF-857T allele); -864/-845 nt (lanes 3, 4 for TNF -857T allele and lanes 5, 6 for TNF -857C allele). An additional high molecular weight band is indicated by an asterisk(*). (b) EMSA competition and supershift experiment with MonoMac6 nuclear extracts and -879/-845(T) radiolabelled probe with 10× and 100× excess of unlabelled NF-κB consensus site (lanes 3, 4) or OCT1 consensus site (lanes 5, 6); or Antibody against NF-κB p50 (lane 7), p65 (lane 8) and OCT1 (lane 9).

FIG. 2

OCT1 interacts with NF-κB in vitro and in vivo. (a) ³⁵S labelled in vitro translated OCT1 was incubated with either GST alone (lane 4) or with p50 or p65Δ-GST-fusion proteins (lane 2, 3) bound to glutathione-agarose beads. 25% of the 35 S labelled OCT1 added to the reactions is shown as Input (lane 1). (b) 35 S labelled in vitro translated full length and truncated OCT1 protein was incubated with matrix-bound p50-GST (lanes 9-12), p65Δ-GST (anes 13-16) fusion proteins or GST alone (lanes 5-8). Input (lanes 1-4) indicates 25% of labelled proteins used in reactions. (c) COS-7 cells were transfected either with an empty RcCMV expression vector (lane 1) or CMV-OCT1-His (lane 2) or the combination of CMV-OCT1-His and CMW-p65 (lane 3) and radiolabelled with 35 S-methionine. OCT1 containing complexes were immunoprecipitated using anti-His Antibody attached to the agarose beads, run on 10% SDS-PAGE and subjected to auto-radiography (left panel). The co-immunoprecipitation of p65 with OCT1 was detected by Western blotting using anti-p65 Antibody (right panel).

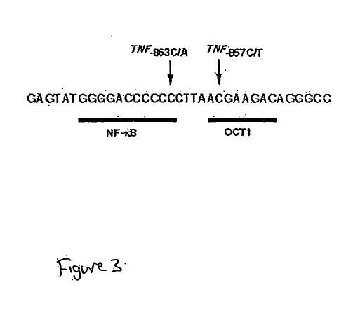

FIG. 3

EMSA probe corresponding to -879 to -845 bp of the TNF promoter. Arrows mark SNP locations, and bars mark putative NF-κB and OCT1 consensus binding sites.

FIG. 4 shows the promoter region of the TNF gene.

### EXAMPLE

Subjects

Northern European Caucasian families were ascertained from the UK, and the diagnosis of CD or UC confirmed using standard criteria (Lennard Jones Scand J Gastroenterol Suppl 170 2-6 (1989)). We genotyped multiplex families (ascertained with two or more siblings affected with IBD) and simplex families (one child affected with IBD). Families with both parents available were genotyped for the transmission disequilibrium test (TDT) analysis, and comprised 101 multiplex families and 355 simplex families containing 127 (multiplex family) and 167 (simplex family) CD trios, 74 (multiplex family) and 178 (simplex family) UC trios, and 10 indeterminate colitis trios. Unrelated cases were also selected from a further 30 multiplex families with only one or no parent(s) available. Healthy unrelated individuals were recruited from the UK Blood Transfusion Service. Informed consent and full ethical approval were obtained.

Genotyping

We isolated genomic DNA from peripheral blood, and performed PCR using primers as described for the TNF -308G/A, TNF -857C/T and TNF -863C/A polymorphisms (Skoog et al, Hum Mol Genet 8 1443-9 (1999)). The TNF -1031T/C polymorphism was amplified with the forward primer (5′-CAGGGGA AGCAAAGGAGAAG-3′) and reverse primer (5′-CGACTTTCATAGCCCTGGAC-3′). PCR products were digested overnight with restriction enzymes (for TNF -308G/A Ncol, TNF -857C/T and TNF -863C/A HypCH4rV, TNF -1031T/C BbsI), and separated by agarose gel electrophoresis. Two investigators, unaware of an individual's affection/pedigree status, called genotypes independently and conflicts were either resolved or the assays were repeated.

Association Analysis

PEDCHECK software was used to check for misinheritance (28), and the TDT calculated by the ASPEX program package (ftp://lahmed.stanford.edu/pub/aspex) for single nucleotide polymorphisms (SNPs) or by an unbiased multilocus haplotype method (Dudridge et al AM J Hum Genet 66 2009-2012 (2000)). Both programs correct for testing multiple siblings from the same family, thus the P value obtained for the TDT is a valid test of association in the presence of linkage. To assess gene-gene interaction of TNF and NOD2 variants for the CD phenotype, we further analysed CD patients who were or were not carriers for common NOD2 variants associated with CD (Arg702Trp, Gly908Arg, Leu1007fsinsC). Unrelated affected individuals (one per family, at random) were compared with healthy controls in a further association analysis (Fisher's exact test), and odds ratios calculated.

TNF Production By Stimulated Whole Blood

Whole blood from healthy controls was collected into sterile tubes with heparin 20 iu/ml, diluted with an equal volume of RPMI 1640 and incubated with or without 10 ng/ml lipopolysaccharide (LPS) from Escherichia Coli 055:B5 (Sigma, Poole, UK) in 5% carbon dioxide at 37° C. Supernatants were harvested at 2, 4 and 8 h after stimulation and TNF levels were measured by ELISA (R&D Systems, Abingdon, UK) as described (Kwiatkowski et al Lancet 336 1201-4 (1990)). Samples were corrected for the unstimulated TNF level at 8 hours, results according to genotype expressed as meanSEM, and differences compared using a two tailed t-test.

DNA Constructs

Human p50 and p65 expressing constructs in Rc/CMV vector (Invitrogen, Groningen, The Netherlands) were previously described (Kuprash et al Oncogene 11 97-106 (1995)). The protein sequences corresponding to amino acids 2-400 of p50 and 2-306 of p65 were recovered by PCR using the appropriate primers and cloned into the bacterial expression vector pGEX-4T-1 (Amersham Pharmacia Biotech, Little Chalfont, UK) encoding the glutathione-S-transferase tag (GST). The (−83)-pGL3 construct (further referred to as (−83)) was generated by PCR amplification asing TNF (−83)-BglII primer (5′-aatagatctGGA AGTTTTCCGCTGG-3′) and vector-specific primer HindIII (5′-AATGCCAAGCTTGGAAGAG-3′) and (−1173)-pGL3 construct as DNA template, and subsequently cloned into HindIII/BglII sites of modified pGL3-basic vector. The region between −922 and −803 bp was amplified by PCR using the primers: forward (F) (Kpnl): 5′-aatggtaccCCACAGCA ATGGGTAGGA-3′ and reverse(R) (SacI): 5′- aatagagctcGGAGGTCC TGGAGGCTC-3′ with TNF promoter wild type and -857T polymorphic mutant as DNA templates. PCR fragments were cloned into KpnI/SacI sites of the (−83) construct.

The sequence corresponding to amino acids 1-742 of human OCT1 was recovered by PCR using the appropriate primers (OCT1 F (BamHI):

5′aatggatccATGAACAATCCGT CAGAAA-3′ and OCT1 R (XhoI): 5′-aatctogagCTGTGCCTTGGAGGCG-3′) and cDNA derived from MonoMac6 cells.

cDNA was cloned into BamHI/XhoI sites of the eukaryotic expression vector pcDNA3 (Invitrogen). The OCT1 deletion constructs were generated by PCR using the same forward primer and three reverse primers and the full length OCT1 as the DNA template:

| | |
|---|---|
| OCT1ΔC (XhoI): | 5′-aatctcgagTGGTGGGTTGATTCT-3′ |
| | (438 amino acids); |
| OCT1ΔPOUH (XhoI): | 5′-aatctcgagTGAGAGGTTCTCTGC-3′ |
| | (357 amino acids); |
| OCT1ΔPOUS (XhoI): | 5′-aatctcgagGGGAGTATCAATTGG-3′ |
| | (276 amino acids); |

PCR fragments were cloned into the pcDNA3 expression vector. All constructs were verified by DNA sequencing.

Protein Extracts and Electrophoretic Mobility Shift Assay (EMSA)

Oligonucleotide probes were radiolabelled with [³²P]-dCTP (Amersham Pharmacia Biotech, Little Chalfont, UK):

−879/−845(C/T) F:
5′agctGAGTATGGGGACCCCCCCTTAA[C/T]GAAGACAGGGCC-3′;
−879/−845(C/T) R:
5′gctGGCCCTGTCTTC[G/A]TTAAGGGGGGGTCCCCATACTC-3′;
−864/−845(C/T) F:
5′-agctCCCTTAA[C/T]GAAGACAGGGCC-3′;
−864/−845(C/T) R:
5′-agctGGCCCTGTCTTC[G/A]TTAA GGG-3′;

879/858 (as κB1 described in Udalova Mol Cell biol 20 9113-9 (2000)).

MonoMac6 cells (10-20×10⁶) were stimulated with 100 ng/ml LPS for 1 h and nuclear extracts were prepared as described in Schreiber et al Nuc Acid Res 17 6419 (1989)). The binding reaction contained 12 mM HEPES, pH 7.8, 80-100 6 mM KCl, 1 mM EDTA, 1 mM EGTA, 12% glycerol and 0.5 μg of poly dI-dC (Amersham Pharmacia Biotech). Protein extracts (1-4 _g) were mixed in an 8 _l reaction with 0.2-0.5 ng of labelled probe (1-5×10⁴ cpm) and incubated at room temperature for 10 minutes. Where indicated, a competitive cold probe corresponding to the consensus binding site for NF-κB (as κB1 described in (24) or OCT-1 (OCT1 F:5′-agctCCCTTAATGCAAATAGG; OCT1 R: 5′-agctCCTATTTGCATTAAGGG) or antibody against NF-κB p65 and p50 or against OCT-1 (Santa Cruz Biotechnology Inc., Santa Cruz, Calif., USA) were added prior to the radiolabelled probe. The reaction was analysed by non-denaturing 5% polyacrylamide gel electrophoresis at 4° C.

Protein-protein Interactions

Proteins were translated in vitro using the TNT quick coupled transcription-translation kit (Promega, Southampton, UK) in a 10 μl reaction containing [³⁵S]-methionine (Amersham Pharmacia Biotech). Glutathione-bound agarose beads containing normalised amounts of p50, p65Δor POU domain GST-fusion proteins, expressed in BL21(DE3)LysS cells (Novagen, Madison, Wis., USA) and purified according to the GST Gene Fusion System manual (Amersham Pharmacia Biotech), were equilibrated in buffer A and subsequently incubated with 3 μl of in vitro translated protein at 4° C. for 2 hours as described in (Yie et al, Embo J 18 3074-89 (1999)). Beads were washed with the same buffer and bound labelled proteins were visualised on 10% SDS-PAGE gel. For in vivo immunoprecipitation experiment COS-7 cells were transfected with CMV-OCT1 and CMV-p65 expressing constructs, labelled with [³⁵S]-methionine in methionine-free medium for 6 hours, and total protein extracts were prepared by lysing cells in RIPA buffer (1×PBS, 1% NP-40, 0.5% Na-deoxycholate, 0.1% SDS) supplemented with protease inhibitors (Boehringer Mannheim Corp., Indianapolis, USA). 20 μl of His-agarose conjugated antibody (Santa Cruz) were added to 100-500 μg of protein extract and incubated at 4° C. over night. Pellets were collected and washed three times with RIPA buffer and once with 1×PBS. Bound labelled proteins were visualised on 10% SDS-PAGE gel, co-precipitated NF-κB p65 protein was visualised by western blotting using anti-p65 antibody (Santa Cruz) and enhanced chemoluminescence system (Amersham Pharmacia Biotech).

Cell Culture, Transfections and Luciferase Assay

MonoMac6 cells were maintained as previously described (Ziegler-Heitbrock et al In J Cancer 41 456-61 (1988)). COS-7 cells were cultured in DMEM supplemented with 10% FBS, 100 U/ml penicillin, 100 mg/ml streptomycin, 0.2 mM L-glutamine and 0.1% glucose. Transient transfections of luciferase gene-reporter and protein expressing were performed on COS-7 cells by using Fugene 6 non-liposomal reagent (Boehringer Mannheim). After transfection cells were incubated for 24 hours prior to harvesting. The luciferase assay was performed using a Turner Designs Luminometer Model 20 (Promega).

Results

TNF Promoter Variants are Associated with CD and UC.

Five common TNF SNP haplotypes were observed, with TNF-1031T/−863C/−857C/−308G the most frequent (54%). The minor TNF-308A and TNF-857T alleles were found on separate unique haplotypes: TNF -1031T/-863C/-857C/-308A and TNF-1031T/-863C/-857T/-308G (founder frequencies 20% and 6% respectively), whereas the minor TNF-1031C allele was observed on two haplotypes: TNF-1031C/-863C/-857C/−308G (6%) or TNF −1031C/−863A/−857C/−308G (13%). TNF-1031C/-863C/-857C/-308G was significantly associated with CD by the TDT (allele transmissions (T) 34: non-transmissions (NT) 17, P=0.02), and a trend towards association seen in CD offspring who carried (T 10: NT 4 P=0.1) or did not carry (T 24: NT 13, P=0.08) a NOD2 mutation. The TNF −857C allele showed significant association with IBD and ulcerative colitis by the TDT. Although the CD phenotype overall did not show association, when we analysed CD affected offspring without mutations in NOD2, significant association with TNF-857C was observed (Table 1). Highly significant associations were also seen with TNF-857C when unrelated cases from the family data were compared to healthy controls (Table 2). The IBD and UC case control association findings, but not the TDT associations, remained significant after Bonferroni correction (×20, highly conservative) for the number of SNPs and phenotypes tested.

Early TNF Induction by LPS is Increased in TNF −857C Homozygotes.

When whole blood from 46 healthy controls was stimulated ex vivo with LPS, TNF production was significantly higher in individuals homozygous for TNF-857C (n=35, mean 65.5±5.7 pg/ml) than in TNF-857T allele carriers (n=11, 46.1±6.5) at 2 hours post LPS stimulation (P=0.03). A non-significant trend towards increased TNF production was also seen at 4 hours (257.9±18.2 versus 229.7±29.1, P=0.4) and 8 hours (407.0±25.0 versus 372.9±30.2, P=0.4) in TNF −857C homozygotes compared to TNF −857T carriers, respectively.

Specific Binding of a High Molecular Weight Protein Complex to the TNF857T Allele.

It has been previously reported that the TNF-863C/A polymorphism is located at a binding site for the transcription factor NF-κB (Udalova et al, supra), and that OCT1 also shows allele-specific binding somewhere in this region (Hohjoh, Genes Immun 2 105-9 (2001)). A fragment spanning bp −879 to −845 of the TNF promoter and containing either the TNF −857C (FIG. 1a, lanes 3, 4) or the TNF-857T (lanes 5, 6) variant was incubated with nuclear extracts derived from the human monocyte cell line MonoMac6, and analysed in an EMSA. There was no constitutive DNA-protein interaction with the TNF-857C allele (lane 3), but the TNF-857C allele formed two inducible complexes with the nuclear extracts derived from the LPS activated cells (lane 4). These complexes were also formed with the shorter DNA fragment (bp −879 to −858) that did not extend to the TNF-857C/T polymorphic site (lanes 1, 2) and were identical to previously identified NF-κB p50-p65 and p50-p50 complexes (Udalova et al, supra). In contrast, the TNF-857T allele formed an additional high molecular weight constitutive complex (lanes 5, 6). This complex was also observed with a shorter DNA fragment (−864 to −845 bp) that did not extend to the NF-κB binding site (lane 7, 8). Thus, the high molecular weight complex was specific to the TNF-857T allele (lanes 5-8) but not the TNF-857C allele (lanes 3, 4, 9, 10).

Identification of the TNF-857T Binding High Molecular Weight Complex as OCT1.

The TNF-857T variant (ATGAAGAC) might represent a potential binding site for the OCT1 transcription factor, as 5 out of 8 nucleotides fit the consensus OCT1 binding sequence (ATGCAAAT) (Fletcher et al, Cell 51 773−81 (1987)). An excess of unlabelled oligoduplex corresponding to the OCT1 consensus sequence or anti-OCT1 antibody was used in an EMSA with the −879/−845 promoter fragment. 10× excess of OCT1 consensus sequence in the binding reaction completely abolished the formation of the high molecular weight complex (FIG. 1b, lane 5), as did the presence of anti-OCT1 antibody (lane 9). In contrast, the excess of a duplex corresponding to the NF-κB site had no effect on the high molecular weight complex (lane 3, 4, 7, 8), but diminished the NF-κB specific complexes at 10× (lane 3) and abolished them at 100× (lane 4). Antibody against NF-κB p50 or p65 had no effect on the high molecular weight complex but resulted in the clearance of NF-κB complexes (lanes 7, 8). Taken together, these findings indicate that the TNF-857T polymorphism permits the binding of OCT1 immediately adjacent to a binding site for NF-κB (FIG. 3).

OCT1 Interacts with NF-κB In Vitro.

The above findings showed that a 34 bp DNA sequence in the distal TNF promoter could bind both OCT1 and NF-κB, raising the question of whether OCT1 might functionally interact with NF-κB, as has been described for OCT1 and various other transcription factors (Zwilling et al Nuc Acid Res 22 1655-62 (1994); Zwilling et al Embo J 14 1198-208 (1995); Wang et al Mol Cell Biol 18 368-77 (1998); Kutoh et al Mol Cell Biol 12 4960-9 (1992) and Kakizawa et al J Biol Chem 274 19103−8 (1999)). To explore this question we generated fusion proteins of glutathione S-transferase (GST) with either NF-κB p50 (p50-GST) or the Rel homology domain (RHD) of NF-κB p65 (p65Δ-GST). The fusion proteins bound to agarose beads were then used to examine whether these specific NF-κB elements could interact with OCT1, in an in vitro pull-down assay. In vitro translated labelled OCT1 protein interacted with both p50-GST and p65Δ-GST, but not with GST alone (FIG. 2a). Neither of the GST-tagged proteins retained a significant amount of in vitro translated control TRAF protein (data not shown).

The POUH Domain is Critical for OCT1 Binding to NF-κB.

The OCT1 DNA binding domain, the POU (for Pit, Oct, UNC), has been shown by three-dimensional structural analysis to consist of a bipartite DNA-binding domain containing POU-specific (POUS) and POU-homeodomain like (POUH) domains tethered together by a hypervariable linker (Cox et al J Bio Mol NMR 6 23-32 (1995)). Three deletion mutants of OCT1 were translated in vitro and used in the pull-down assay with either matrix bound p50-GST or p65Δ-GST (FIG. 2b). The C-terminal domain deletion mutant had no effect on the interaction (lanes 10, 14), but the presence of an intact POUH domain was essential for the interaction with both p50 and p65 subunits of NF-κB (lanes 11, 12, 15, 16). DNA binding affinity of a truncated OCT1 protein lacking the POUH domain was only slightly decreased compared to the full-length protein (data not shown. A reverse pull-down experiment was performed with a matrix bound fusion protein of GST and the OCT1 POU domain consisting of both POUS and POUH. In vitro translated p65Δ interacted with POU-GST but not with GST alone (data not shown).

NF-κB p65 and OCT1 are Capable of Interacting In Vivo.

The observation that OCT1 can bind to NF-κB in vitro does not prove that this interaction can take place in the intracellular environment of mammalian cells. To explore this question, COS-7 cells were co-transfected with NF-κB p65 and OCT1 expressing plasmids and all newly synthesised proteins were labelled with [³⁵S]-methionine. The Oct-1 construct was His-tagged. Total proteins were extracted and Oct-1 containing complexes were precipitated with anti-His antibody attached to agarose beads. The immunoprecipitated complex was then run under reducing conditions on SDS-PAGE, showing a radiolabelled band of ˜90 kDa corresponding to Oct-1 (FIG. 2c, left). When the electropheresed product was examined by Western analysis using anti-p65 antibody, a band of the expected size for NF-κB p65 was observed, demonstrating a co-immunoprecipitation of p65 with OCT1 (FIG. 2c, right). Taken together, these findings indicate that NF-κB p65 can physically interact with Oct-1 through its POU domain in vitro and in vivo.

Reporter Gene Analysis in COS-7 Cells

To explore the potential functional role of the TNF-857C/T polymorphism, and how this might relate to an interaction between NF-κB and OCT1, we performed a series of transient co-transfection experiments in COS-7 cells. We generated luciferase gene-reporter constructs containing the two allelic variants (TNF-857C or TNF-857T) of the distal segment of TNF promoter (between −922 and −803 bp) linked to the TNF minimal promoter (−83). These constructs were each transfected into COS-7 cells with or without NF-κB p65 and p50 expressing plasmids. Reporter gene expression was increased in the presence of NF-κB, by a similar amount for TNF-857C and TNF-857T (respectively 2.8-fold and 2.9-fold inducibility). When an OCT1 expression construct was co-transfected along with NF-κB, luciferase gene expression was equally diminished for TNF-857C and TNF-857T (respectively 1.0-fold and 1.1-fold inducibility). When OCT1 was expressed without NF-κB, a modest increase in luciferase activity was seen for TNF-857T but not for TNF-857C (respectively 1.9-fold versus 0.8-fold inducibility).

Discussion

We aimed to study genetic variation in a positional and functional candidate gene for IBD, TNF, and explore the functional significance of any disease-associated polymorphisms. Recently variants in NOD2 have been shown to be associated with CD (Hugot et al Nature 411 599-603 (2001) and Ogura et al J Biol Chem 276 4812-4818 (2001)), and cells transfected with 3′ NOD2 deletion mutants show increased NF-κB activation, which would normally lead to downstream TNF and cytokine expression (Ogura et al, supra; and Ogura et al Nature 411 603-6 (200)). Our data suggested that about a fifth of CD was attributable to common NOD2 mutations, and we postulated that genetic variation in TNF expression might also play a role in IBD pathogenesis. Although associations with TNF promoter polymorphisms have been reported for other diseases, there have been no published papers examining the role of the TNF-1031T/C, TNF-863C/A or TNF-857C/T variants in Caucasian IBD. In our analysis CD, but not UC or IBD as a whole, was associated with TNF-1031C/−863C/−857C/−308G. No evidence for epistatic interaction with NOD2 variants was observed. Association was not seen at the single marker level for either TNF-1031 T/C or TNF863C/A, and it may specifically be the TNF-1031C/−863C/−857C/−308G haplotype that confers a functional effect. A negative association with this haplotype was observed in Japanese CD patients (Kawaski et al Genes Immun 1 351-357 (2001)), but the numbers of CD patients who carry the haplotype in both the Japanese and our Caucasian study were small. Thus whilst it is possible that TNF-1031C/−863C/−857C/−308G has different effects in Japanese and Caucasian CD, these results may also be due to statistical error or population specific linkage disequilibrium with other MHC variants. Further studies in separate populations are necessary to clarify the significance of these findings. Nevertheless, allele NF-κB binding occurs at the TNF-863C/A site. In monocytes, TNF-863C binds both the p50-p65 and p50-p50 dimers of NF-κB whereas the single base change in TNF-863A specifically inhibits NF-κB p50-p50 binding (Udalova, Supra).

Transmission disequilibrium testing demonstrated association of TNF-857C with IBD overall and with UC (that is, the haplotype TNF-1031T/−863C/−857T/−308G protects against disease development). Interestingly, when a subset of CD patients carrying disease associated variants in a known susceptibility gene (NOD2) was removed from the analysis, association of TNF-857C with CD was then observed. TNF-857C and NOD2 variants therefore act independently to confer CD susceptibility. TDT is more robust than case-control analysis in the presence of population stratification, of relevance here due to the observation of higher TNF-857T allele frequencies in other populations (Negoro et al Gastroenterol 117 1062-8 (1999); and McCusker et al Lancet 357 436-9 (2001)). A positive result from TDT analysis therefore carries greater weight than a similar result from a case-control study, however the TDT is less powerful because only heterozygous parents are informative (Cardon et al Nature Reviews Genetics 2 91-99 (2001)). The associations of TNF-857C seen in the TDT analysis were confirmed, and of greater significance, when healthy controls were compared to unrelated cases drawn from the family data. Only a fraction of families were informative for the TDT analysis (123 heterozygous parental allele transmissions/non-transmissions to IBD affected offspring) in comparison to the unrelated cases (IBD, total 952 alleles). Independent support for the TNF-857C associations identified by the TDT is therefore provided by the healthy controls, and the majority of the case population. When the segment of DNA containing the TNF −857 polymorphism is incubated with nuclear extracts from human monocytes, the predominant TNF producing cells in IBD, we find evidence of specific binding to the transcription factor OCT1, but only in the presence of the TNF-857T allele. This is consistent with recently published data on OCT1 binding to this part of the TNF promoter region in B cells (Huhjoh et al Genes Inmun 2 105-9 (2001)). Previous work from this laboratory has identified a complex pattern of NF-κB interactions at the adjacent TNF-863 polymorphism, and this allowed us to map OCT1 binding to the region spanning the TNF-857 site, immediately adjacent to the NF-κB binding site, as illustrated in FIG. 3. OCT1 is a broadly expressed transcription factor that is known to acquire cell-specific activating properties through interaction with other transcription factors but has not previously been reported to interact with NF-κB. One extensively studied example of such an interaction is the herpes simplex virus (HSV) trans-activator VP16, that upon infection associates with OCT1 to form a multiprotein-DNA complex activating the transcription of HSV immediate-early promoters (Herr et al Cold Spring Harb Symp Quant Biol 63 S99-607 (1998)). We show here that OCT1 can interact with the Rel homology domain of NF-κB and that this interaction maps to the POU domain of OCT1, with POUH being critical for the interaction. Both domains are DNA-binding, therefore the protein-protein interaction could affect the binding of these factors to the TNF promoter. The association of TNF-857C with IBD in Caucasians is particularly interesting, as we demonstrate TNF production in whole blood to be increased in TNF-857C homozygotes and increased TNF production has previously been shown in human IBD. We observe (1) that this part of the TNF promoter region contains adjacent binding sites for OCT1 and for NF-κB; (2) that OCT1 is capable of interaction with NF-κB; (3) that OCT1 binding to this region is abolished in the presence of the TNF-857C allele; (4) that allele specific binding of OCT1 to the TNF promoter is reported at the TNF-376G/A site, which alters constitutive TNF expression and is associated with susceptibility to cerebral malaria (Knight et al Nat Genet 22 145-50 (1999)). Taken together, these findings raise the possibility that allele-specific OCT1 binding to the TNF-857C/T region serves to suppress certain types of TNF response in the gut, thereby reducing the likelihood of EBD. Our reporter gene experiments in COS-7 cells provide partial support for this hypothesis, in that OCT1 appeared to suppress the NF-κB responsiveness of this promoter segment but in this system we did not observe any difference between the TNF-857T and TNF-857C alleles. Here we have used COS-7 cells as a reductionist model to examine the potential interactions of NF-κB with OCT1 in this part of the TNF promoter region, and other laboratories have reported variable effects of the TNF-857 polymorphism on reporter gene expression in other cellular models (Uglialoro et al Tissue Antigens 52 359-67 (1998) and Higuchi et al Tissue Antigens 51 605-12 (1998)). However, all are artificial in vitro systems that provide a very limited insight into how TNF is regulated in vivo in specific tissues (such as the gut) under the influence of biologically relevant stimuli (Papadakis et al Gastroenterol 119 1148-57 (2000)). Ideally the functional effects of the TNF-857C/T polymorphism in EBD should be studied in lamina propria mononuclear cells, but this is technically difficult, as these cells are refractory to plasmid transfection. We are currently exploring adenoviral infection as an alternative method of gene-reporter delivery (Udalava et al, Supra). Our genetic data identifies the TNF-857C allele as a marker of susceptibility to IBD in the UK population, but this variant could be either a true disease allele or a marker allele in linkage disequilibrium with a neighbouring functional polymorphism. The association between the TNF-857C allele and TNF production by whole blood is consistent with it being a functional variant, and we have identified a possible molecular mode of action, through allele specific binding of OCT1 to the distal TNF promoter and the interaction at this site between NF-κB and OCT1.

1. A method of determining susceptibility of a Caucasian subject to Crohn's Disease, the method comprising screening the genetic material of the subject for the TNF-1031 C/−863C/−857C/−308G haplotype.

2. A method according to claim 1 wherein presence of the TNF −1031C/−863C/−857C/−308G haplotype confers susceptibility to Crohn's Disease.

3. A method of confirming the diagnosis of a Caucasian subject as having Crohn's Disease, the method comprising screening genetic material of the subject for the presence of the TNF-1031 C/−863C/−857C/−308G haplotype.

4. A kit for use in determining the susceptibility of a Caucasian subject to Crohn's Disease, the kit comprising means for screening for the TNF-1031C/−863C/−857C/−308G haplotype, and a key indicating the correlation between genotype and susceptibility to Crohn's Disease.

5. A method of preventing or treating Crohn's disease in a Caucasian subject, the method comprising introducing into the subject genetic material comprising one of the TNF-1031T allele, the TNF-863T allele, the TNF-857T allele, and the TNF-308A allele.

6. A method according to claim 5 wherein genetic material comprising the TNF-1031T/−863T/−857T/−308A haplotype is introduced.

7. A method of manufacturing a medicament for the prevention or treatment of Crohn's disease in a Caucasian subject said method comprising incorporating genetic material comprising one of the TNF-1031T alleles, the TNF-863T alleles, the TNF-857T allele, and the TNF-308A allele, into a medicament, wherein said medicament is effective to prevent or treat Crohn's disease in a Caucasian subject.

8. A method of manufacturing a medicament for the prevention or treatment of Crohn's disease in a Caucasian subject said method comprising incorporating genetic material comprising the TNF-1031T/−863T/−857T/−308A haplotype into a medicament, wherein said medicament is effective to prevent or treat Crohn's disease in a Caucasian subject.

9. A method of determining the susceptibility of a subject to Inflammatory Bowel Disease, the method comprising screening the genetic material of the subject to determine which allele of the TNF-857C/T polymorphism is present.

10. A method according to claim 9, wherein the Inflammatory Bowel Disease is Ulcerative Colitis.

11. A method according to claim 9, wherein the Inflammatory Bowel Disease is Crohn's Disease.

12. A method according to claim 11, further comprising screening the genetic material of the subject to determine which allele of the NOD2 polymorphism that is associated with Crohn's disease is present in the genetic material of the subject.

13. A method according to claim 9, wherein the method is performed on subjects identified as having the NOD2 Arg 702Trp, Gly 908Arg or leu10007 FinsC allele.

14. A method for determining the response of a patient to treatment, the method comprising screening the genetic material of a subject to determine which allele of the TNF-857C/T and/or NOD2 polymorphisms are present.

15. A kit for use in determining the susceptibility of a subject to Inflammatory Bowel Disease or for determining the response of a patient to treatment, the kit comprising means for determining which allele of the TNF-857C/T and/or NOD2 polymorphisms are present, and a key correlating the presence of an allele with the susceptibility to disease.

16. A method of treating a subject determined as being susceptible to disease according to the method of claim 1, comprising administering an agent capable of preventing TNF production.

17. An agent capable of preventing TNF production for use in a method of treating a subject for disease, wherein the subject has been identified as being susceptible to disease according to the method of claim 1.

18. A method of manufacturing a medicament, said method comprising incorporating an effective amount of an agent capable of preventing TNF production into a medicament wherein said medicament comprising said agent is effective to prevent TNF production in a subject determined to be susceptible to disease according to a method of claim 1.

19. An agent which is capable of modulating the activity of OCT1 and/or NF-κB.

20. An agent according to claim 19 which modulates the interaction between OCT1 and NF-κB.

21. An agent according to claim 20 which modulates the binding of OCT1 to DNA.

22. An agent according to claim 19 which is capable of binding to the T allele of the TNF-857C/T polymorphism.

23. An agent according to claim 22 which is capable of interacting with the Rel homology domain of NF-κB.

24. An agent according to claim 19, which is capable of inhibiting the activity of NF-κB.

25. An agent according to claim 19, wherein the agent is a variant of OCT 1.

26. An agent according to claim 19 for use in treating or preventing Inflammatory Bowel Disease in a subject.

27. A method of manufacturing a medicament, said method comprising incorporating an agent capable of modulating the activity of OCT1 or NF-κB into a medicament, wherein said medicament comprising said agent is effective to treat or prevent Inflammatory Bowel Disease in a subject.

28. The method of claim 5 comprising introducing into the subject genetic material comprising the TNF-1031T allele, the TNF-863T allele, the TNF-857T allele, and the TNF-308A allele.

29 The method of claim 7 wherein the genetic material comprises the TNF-1031T allele, the TNF-863T allele, the TNF-857T allele, and the TNF-308A allele.
